(19) United States
(12) Patent Application Publication
(10) Pub. No.: US 2003/0092015 A1
(43) Pub. Date: May 15, 2003

(54) Method for screening and/or identifying factors that bind to nucleic acids

(76) Inventors: Anne-Marie Larose, Montreal (CA); Pierre Rousseau, Longueuil (CA); Benoit Leblanc, Montreal (CA); Rino N. Camato, St-Leonard (CA)

Correspondence Address: MERCHANT & GOULD PC, P.O. BOX 2903, MINNEAPOLIS, MN, 55402-0903, US

(21) Appl. No.: 10/023,318

(22) Filed: Dec. 14, 2001

(30) Foreign Application Priority Data
Dec. 27, 2000 (CA) 2 327 561

(51) Int. Cl. C12Q 1/68; C12P 19/34

(52) U.S. Cl. 435/6; 435/91.2; 435/270

(57) ABSTRACT

The invention provides a novel method for the screening and for the identification of nucleic acid binding factors (NABFs) or nucleic acid binding elements (NABEs) that are differentially active between two phenotypically different cell lines (normal and modified); in a particular cell type before and after a given treatment; or between two completely heterologous cell lines. The invention also provides methods for the simultaneous analysis of the effect of given compounds on multiple DNA-protein interactions. It further provides for the analysis of compounds that are nucleic acid binding factor analogs and compounds that selectively bind cis-acting nucleic acids.

### FIELD OF THE INVENTION

The present invention relates to a method for screening and/or identifying factors that bind to nucleic acids. Specifically, the present invention provides a novel method for screening and/or identifying differentially active nucleic acid binding factors (NABFs) or nucleic acid binding elements (NABEs) using DNA microarray technology.

### BACKGROUND OF THE INVENTION

Nucleic acid binding proteins are involved in a variety of cellular processes ranging from transcription and replication to recombination and viral integration. Transcription factors are proteins that bind to specific sequences of DNA and influence the transcription of the DNA into mRNA. Some of these factors directly participate in the transcription process by activating or inhibiting the transcription and regulate the synthesis of proteins needed by cells to function, to adapt, to respond or to differentiate. Some of these proteins have to be transcribed in a constitutive manner (being essential to basic cell function) while others are only synthesized in response to specific stimuli, or when the cells are in a certain condition, such as a pathological state. Modulation of gene expression by the cell environment occurs when external signals are sensed by membrane-based receptors and transduced through the plasma membrane, triggering cascades of enzymatic reactions which ultimately induce post-translational modifications of transcription factors. These modifications can positively or negatively affect the capacity of transcription factors to bind to their cognate recognition sequences.

Up until now, the sequence-specific binding of proteins to DNA has been studied by a variety of assays, among which the electrophoretic mobility shift assay (EMSA) has been the most frequently used. EMSA is a useful method for visualizing specific interactions between DNA-binding proteins and DNA. In this assay, the DNA binding proteins slow down the migration of labelled oligonucleotides as they travel through an electrophoretic gel in non-denaturing conditions (Ausubel et al., 1993). Typically, ³²P-labelled DNA probes containing the sequences bound by the proteins of interest are used in mobility shift assays. A non-radioactive method using DNA labelled with digoxygenin-dUTP has also previously been described (Suske et al., 1989).

Other methods are also used to analyse DNA-protein interactions like the ABCD assay (Glass et al, 1987), the antibody-based DNA binding assay (Furlow et al, 1993), gel filtration assays (Peale et al, 1988; Shupnik and Rosenzweig, 1991) and the microtiter well assay (Ludwig et al. 1990). Due to the nature of these techniques, they are not suitable for use in large-scale analyses because each assay involves an individual reaction linked to specific components, such as antibodies, that must be added to the reaction. All of these techniques are also labour-intensive.

In U.S. Pat. No. 6,100,035, Cistem Molecular Corporation describes a method for identifying nucleic acid molecules that contain cis-acting nucleic acid elements. In this method, as in other similar methods described in the literature, the identification of nucleic acid binding elements (NABEs) involved several steps, including DNA amplification and sequencing. Moreover, methods such as these do not provide information regarding the direct identification of differentially active NABEs.

Microarrays may be used as a high-throughput tool for the large-scale identification of nucleic acid binding factors (NABFs) or NABEs. They have been used to identify the interaction of proteins with segments of DNA in entire genomes, using an enrichment of protein-bound DNA by chromatin immunoprecipitation as a first step (Ren et al., 2000). However, such assays rely heavily on the quantitative reproducibility of the immunoprecipitation.

### OBJECT OF THE INVENTION

The general object of the present invention is therefore to provide an improved method for the screening and/or identification of nucleic acid binding elements (NABEs) and nucleic acid binding factors (NABFs).

### SUMMARY OF THE INVENTION

In accordance with the present invention, there is provided a method that allows the rapid identification of NABEs or NABFs that are differentially active in cells that have undergone particular treatments or that are phenotypically different.

NABEs can be made from synthetic oligonucleotides, PCR amplified DNA, cloned DNA or a combination thereof. Each contains a unique consensus nucleic acid binding element already known or a promoter sequence with putative nucleic acid binding element. One of the strands of each of the NABEs used in the nucleic acid binding assay is also used as NABE-probe (NABE-p), which is individually spotted onto glass slides or another suitable solid support to make the arrays that are later used to identify particular NABEs from a mixed pool. Pools of NABEs are incubated in conditions conducive to binding with protein extracts containing the NABFs studied. Protein extracts from two sources (normal and abnormal cells, healthy and diseased cells, or treated and untreated cells, for example) are used independently on equivalent pools of NABEs. In both cases, NABEs bound by NABFs, or NABE-NABF complexes, are separated from unbound NABEs. This step can be performed with a standard method, such as a preparative EMSA. NABEs are then purified from the proteins present in the NABE-NABF complexes by, for example, cutting the NABE-NABF complexes out from a gel matrix or electroeluting them, and extracting the NABEs from the NABFs by standard nucleic acid purification techniques such as phenol extraction. They are then labelled with fluorescent dyes. NABEs derived from experiments performed with different nuclear extracts are labelled using different dyes in order to trace them back to the proper extract. For example, Cy3-dCTP can be used to label purified NABEs previously bound by NABFs from a protein extract made from untreated cells and Cy5-dCTP to label those previously bound by NABFs from an extract made from cells treated with a drug. Finally, labelled NABEs are cohybridized to a DNA microarray chip in order to quantify the NABEs recovered from each experiment. Cohybridization allows the immediate comparison of binding activity of NABFs to NABEs in each of the two cases. In cases where the NABEs consist of promoter sequences or genomic sequences, the present invention allows the identification of cis-acting promoter elements.

Although described for use in conjunction with microarrays, the principle of this invention can also apply with any type of solid support for the bound oligonucleotides recognizing the recovered NABEs. An example of an alternate system would be to use oligos bound to microbeads, as per the technology of the Luminex corporation (Austin, Tex.). In such an embodiment, the technique would make use of a number of microscopic beads, each labelled with a certain proportion of two fluorescent agents allowing their individual recognition by the appropriate type of detector. Each of the beads would then be coupled with one of the oligonucleotides capable of hybridizing with the NABEs recovered after the EMSA step of the operation. The aforementioned EMSA would have to be performed with double-stranded DNA fragments (oligonucleotides or otherwise) which could be coupled to a fluorescent agent, such as a biotin tag which might be recognized by a streptavidin-phycoerythrin complex. In a typical experiment using this variation of the technique, the biotinylated NABEs would be recovered after the EMSA step and would be incubated with the streptavidin-phycoerythrin complex and the microbeads before being analyzed by the detector to find out which NABE hybridized to which bead.

More specifically, in one embodiment the present invention provides a method for screening nucleic acid binding elements (NABEs), comprising:

(a) contacting nucleic acid binding factors (NABFs) with NABEs under conditions to promote specific binding interactions therebetween;

(b) identifying complexes formed between the NABEs and the NABFs (NABE-NABF complexes);

(c) separating the NABF from the NABE-NABF complexes to obtain NABEs that bind to NABFs;

(d) marking the NABEs obtained in (c) (marked NABEs);

(e) contacting the marked NABEs with probes of known nucleic acid binding elements (NABE-ps) bound to a support under conditions to promote hybridization therebetween; and

(f) analyzing the hybridization in (e) in order to identify the marked NABEs.

In an alternative embodiment, the invention provides a method for screening nucleic acid binding elements (NABEs) that are differentially active in modified cells, comprising:

(a) contacting nucleic acid binding factors (NABFs) with NABEs derived from a modified cell under conditions to promote specific binding interactions therebetween;

(b) identifying complexes formed in (a) between the NABEs and the NABFs (NABE-NABF complexes);

(c) separating the NABFs from the NABE-NABF complex to obtain NABEs that bind to NABFs;

(d) marking the NABEs obtained in (c) (marked NABEs);

(e) contacting the marked NABEs with probes of known nucleic acid binding elements (NABE-ps) bound to a support under conditions to promote hybridization therebetween; and

(f) analyzing the hybridization in (e) in order to identify the marked NABEs that are differentially active in the modified cell.

Relying on this latter embodiment, the method of the present invention may further comprise:

(g) contacting the NABFs with nucleic acid binding elements derived from a non-modified cell (non-modified cell NABEs) under conditions to promote specific binding interactions therebetween;

(h) identifying complexes formed in (i) between the non-modified cell NABEs and the NABFs (non-modified cell NABE-NABF complexes);

(i) separating the NABF from the non-modified cell NABE-NABF complex to obtain non-modified cell NABEs that bind with NABFs;

(j) marking the non-modified cell NABEs obtained in (i) with a marker that is different than the marker used to mark the modified cell NABEs (marked non-modified cell NABEs);

(k) contacting the marked non-modified cell NABEs with the NABE-ps bound to a support under conditions to promote hybridization; and

(l) comparing the amount of hybridization in (v) with the amount of hybridzation in (e) in order to identify the differentiated activity of the marked NABEs derived from the modified cell.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

In the appended drawings:

FIG. 1: Arraying of individual NABE-p for the microarray fabrication;

FIG. 2: Schematic representation of the analysis of differentially-bound NABEs by DPA;

FIG. 3: Identification of differentially bound NABEs. A ratio of 1 reflects an equivalent DNA-binding activity for a given factor in both conditions tested. A ratio <1 signals a reduced DNA-binding activity while a ratio >1 reflects an increase in such activity. No ratio (no signal) means no factor bound in either condition;

FIG. 4: A hybridized NABE-p array with Cy5 end-labelled NABEs. 300, 90 and 9 pg of each NABE-p are spotted in duplicata. Non specific oligos are also spotted as negative control;

FIG. 5: Hybridization of NABEs retrieved from a shifted NABE-NABF complex to a NABE-p microarray; and

FIG. 6: Cohybridization of NABEs incubated in K562 cells nuclear extracts and recovered by EMSA to a NABE-p microarray. NABEs incubated with an extract from untreated cells were labelled with Cy3 (green) while NABEs incubated with an extract from TPA-treated cells were labelled with Cy5 (red). Yellow reflects equivalent quantities of both types of NABEs.

### DESCRIPTION OF THE PREFERRED EMBODIMENT

Glossary

In order to understand the invention more clearly, a general definition of certain terms is given below.

CONSENSUS SEQUENCE: A consensus nucleotide sequence refers to a particular string of nucleotides representing what would be a typical binding site for a specific nucleic binding factor. It is often deduced by comparing the sequence of many experimentally observed binding sites for the factor of interest.

Cy3, Cy5: Non-radioactive fluorescent dyes from Amersham Pharmacia Biotech that are widely used for labelling DNA in microarray experiments.

EMSA: Electrophoretic mobility shift assay.

HYDRIDIZATION: The process of joining two complementary strands of DNA, or one strand each of DNA and RNA, to form a double-stranded molecule. NABE: Nucleic acid binding element. The term “nucleic acid binding element” (NABE) is used to designate all specific sequences of nucleotides in a nucleic acid (DNA or RNA) that can be specifically recognized by and be bound by a NABF.

NABF: Nucleic acid binding factor. The term “nucleic acid binding factor” (or NABF) is used to designate all factors capable of recognizing, and binding to, a nucleic acid in a specific fashion.

NABE-NABF: Complex involving a nucleic acid binding element (NABE) bound by a nucleic acid binding factor (NABF).

NABE-p: NABE-probe. A nucleic acid bearing a sequence complementary to at least one of the strands of the NABE so that the NABE-p can hybridize with the NABE.

OLIGO: An oligo, short for “oligonucleotide” is a sequence formed of at least two nucleotides. While the term oligonucleotide is generally used in the art to denote smaller nucleic acid chains, and “polynucleotide” is generally used to denote larger nucleic acid chains including DNA or RNA, chromosomes or fragments thereof, the use of one or the other term herein is not a limitation or description of size unless specified.

SEQUENCE: A sequence (e.g. sequence, genetic sequence, polynucleotide sequence, nucleic acid sequence) refers to the actual enumerated bases (ribo- or deoxyribonucleotides) present in a polynucleotide strand reading from the 5′ to 3′ direction.

In general, the present invention provides a method for screening factors (usually proteins) that bind to nucleic acids (RNA or DNA). Among these factors, the most studied are known as transcription factors, proteins which bind DNA in expression-control regions usually designated by their functional role, such as promoters and enhancers. Transcription factors modulate gene expression by binding to particular nucleic acid elements that they recognize.

The present invention also provides a method for screening factors in cells that have been treated or altered, such as cells treated with a substance or such as cancerous cells, and comparing the nucleic acid binding activity found therein to that found in appropriate control cells.

In practice, the assay consists of a multiplicity of electromobility shift assays (EMSAs) performed at the same time in a single large well of a gel, and their subsequent discrimination, identification and comparison on a microarray or any other solid support capable of carrying a large number of nucleic acid probes. In the case of control cells, a nuclear or cellular extract is performed from the cells as if for a standard EMSA aiming to analyze a single NABE-NABF interaction. This extract is then incubated, in conditions conducive to the formation of such complexes, with a pool of nucleic acid fragments of lengths which are both similar and appropriate for a standard EMSA analysis. The complete binding reaction is then submitted to electrophoresis separation, as in a standard EMSA. The pool of NABEs bound by NABFs will have a retarded migration compared to that of the unbound NABEs and will be recovered by electroelution from the gel's matrix and purified so as to get rid of the bound proteins by methods known to those of skill in the art. The same operation is performed independently using an equivalent quantity of the same pooled NABEs and an extract made from the tested cells, be they cells treated with a drug and being compared with untreated cells, diseased cells being compared to healthy cells, or any other sort of cells being compared to a suitable control. The NABEs purified from the NABE-NABF complexes recovered from the gel in this case will be kept separate from the NABEs purified from the previous step involving control cells.

The two pools of NABEs thus recovered are then independently labelled, by using, for example, terminal transferase and deoxyribonucleotides with a red-fluorescent label for one of them and a green-fluorescent label for the other. Since the labels are distinguishable from each other, a direct comparison of the NABE-binding activity present in both control and tested cells may be made following the hybridization of the labelled NABEs to a microarray or any other solid support carrying appropriate probes with sequences complementary to the sequences of the individual NABEs.

The results provide an insight into the activity of NABFs derived from modified or test cells as compared with that of NABFs derived from control or reference cells.

Materials and Methods

Array construction

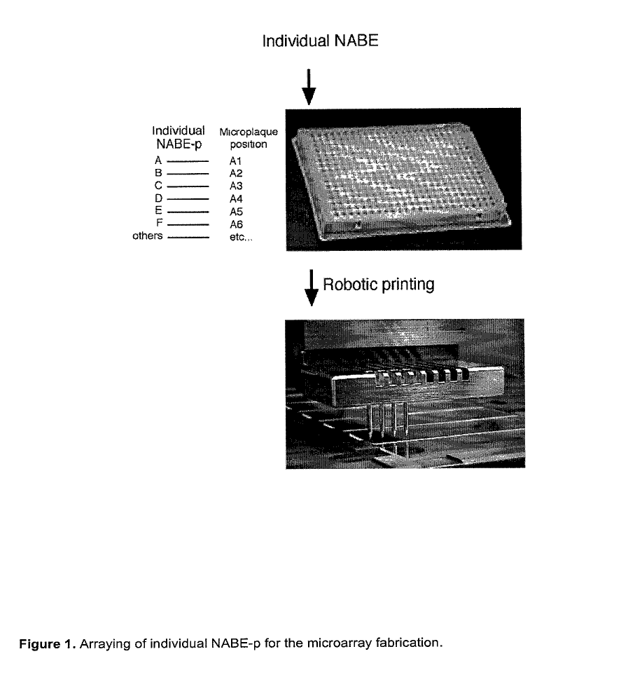

As illustrated in FIG. 1, for each NABE a corresponding NABE-probe (NABE-p) is needed for the array. These NABE-ps can be made from synthetic oligonucleotides, PCR amplified DNA, cloned DNA or a combination thereof. NABE-ps are individually spotted onto the glass slide. The length of a NABE-p may vary considerably (from about 20 bases to more than about 1000 bases). Where double-stranded DNA is the source for NABE-ps, a denaturating step prior to use must be performed. When short DNA fragments or oligos are used, the NABE-ps can be modified at extremity to allow end-attachment to the slide and to introduce a certain distance between the slide and the specific sequence. For example, 5-amino modified oligos with linkers consisting of a chain of twelve carbons atoms have been found to be useful. The NABE-ps may include modified nucleotides. Modified internucleotide linkages are useful in probes comprising deoxyribonucleotides and oligonucleotides to alter, for example, hybridization strength and resistance to non-specific degradation and nucleases. The links between nucleotides in the probes may include bonds other than phosphodiester bonds, such as peptide bonds. Modified internucleotide linkages are well known in the art and include methylphosphonates, phosphorothioates, phosphorodithionates, phosphoroamidites and phosphate ester linkages. Dephospho-linkages between nucleotides, also known as bridges, include siloxane, carbonate, carboxymethyl ester, acetamidate, carbamate, and thioether bridges. “Plastic DNA” comprised of N-vinyl, methacryloxyethyl, methacrylamide or ethyleneimine internucleotide linkages can also be used in probes (see Uhlmann and Peyman, 1990). “Peptide Nucleic Acid” (PNA) is particularly useful because of its resistance to degradation by nucleases and because it forms a stronger hybrid with natural nucleic acids (Orum et al. (1993); Egholm et al. (1993) herein incorporated by reference). The solid support may be glass with an appropriate coating or any other material suitable for DNA microarray experiments, allowing DNA attachment and having a low or non-existent flurorescent background).

Robotic printing of microarrays can be performed using one of the several arrayers available on the market.

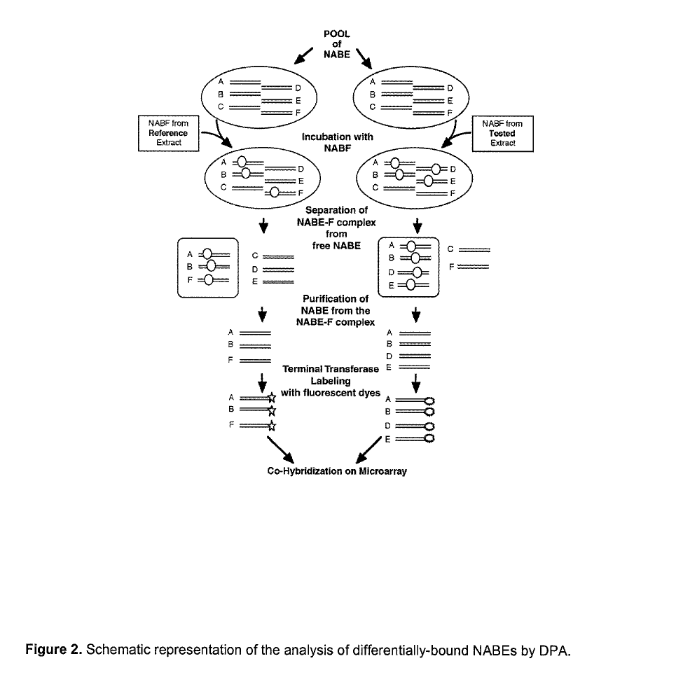

Differentially bound NABE preparation (target preparation) (FIG. 2)

A NABE is a nucleic acid polynucleotide sequence containing a known consensus binding sequence or putative binding sequence. Examples of DNA consensus binding sites are available from many venues, such as the web site http://transfac.gbf-braunschweig.de/TRANFAC. DNA binding site sequences can be also obtained from experimental results or from the literature. Promoter sequences, or genomic fragments or other sequences likely to contain binding sites, can also be used as NABEs. Instead of performing a binding reaction with an individual NABE, as is typically done in the course of an EMSA assay, the present invention uses pools of NABEs for the binding reaction (i.e., more than two different NABEs are used in the same binding reaction).

The NABFs used for the binding reaction with the NABEs do not have to be cloned, purified or labelled. Crude protein extracts or nuclear protein extracts are good sources of NABFs. There are several protocols known in the art that can be used to extract proteins in such a way that the different NABFs present in an extract maintain their nuclear acid binding activities.

The incubation conditions that will allow the binding of NABEs by NABFs are well known in the art. For example, reaction mixtures containing the NABEs, the protein extracts (NABFs), the binding buffer (10 mM Tris-HCl pH7.9, 50 mM KCl, 1 mM Dithiothreitol and 5% (v/v) glycerol) and 1 μg of poly(dl-dC) are incubated for 20 minutes at 4° C.

The EMSA can be performed to separate the bound NABEs (NABE-NABF complexes) from the free NABEs. Typically, following the incubation of the NABE-NABF binding reaction, each mixture (one with the first type of protein extract that is to be analyzed and the other with the second type) is loaded onto a 5% polyacrylamide gel in 0.5×TGE (25 mM Tris base, 200 mM glycine, 1 mM EDTA) and electrophoresed.

Two types of controls are also loaded for each separation. The first one is a migration standard: on either side of each gel, an EMSA reaction using a radioactively labelled mixture of the NABE used is loaded in order to visualize the position of the shifted bands by exposure to an X-Ray film once the migration has been stopped. (Since the NABEs used for the later hybridization to the microarray are not radioactive, their position would be difficult to ascertain without such a standard.) Alternatively, bromophenol blue can be loaded onto separate lanes and used as a visual marker for the localization of the bound NABE-NABF complex versus free NABE, since the free NABEs (smaller than 100 pb) migrate faster than bromophenol blue in the conditions described. The second control, for its part, aims to evaluate the recovery of DNA from the gel : an independent NABE-NABF binding reaction is set up and loaded in equal quantities on the two EMSA gels. The recovery of this particular NABE from each gel should be equivalent; if it is not, a corrective factor can then be established to account for the discrepancy. Once the position of the bound NABEs on the gel is established, the portion of the gel where these NABEs are located is excised and electroeluted. The free NABEs are then purified using phenol-chloroform (1:1) according to standard protocols, and ethanol-precipitated. The NABEs are then resuspended in a solution suitable for the labelling reaction.

NABEs can be labelled by several methods using appropriate labelling molecules. For example, NABEs can be end-labelled by terminal transferase incorporation of a labelled nucleotide. Purified NABEs from the first sample and from the second sample must be labelled with different molecules so they can be distinguished.

Hybridization

Prior to hybridization, the DNA microarray wafer must be blocked or inactivated in order to prevent the non-specific binding of labelled target to the slide, which could deplete the target and produce a high background at the visualization step. This blocking step also has the advantage of washing unbound DNA from the slide prior to the addition of the probe. Any DNA washed from the surface during hybridization competes with DNA bound to the slide; as the kinetics of solution hybridization are much more favorable than for surface hybridization, this can dramatically decrease the fluorescent signal measured from the microarray.

Slides should be used immediately following the prehybridization or blocking step. There are several protocols known in the art that can be used for the hybridization and washing steps to obtain high specificity while minimizing background noise. At this step, the labelled NABEs obtained from the two samples compared (each labelled with a different fluorescent molecule) are mixed together to perform a co-hybridization using the DNA microarray.

Data collection and analysis

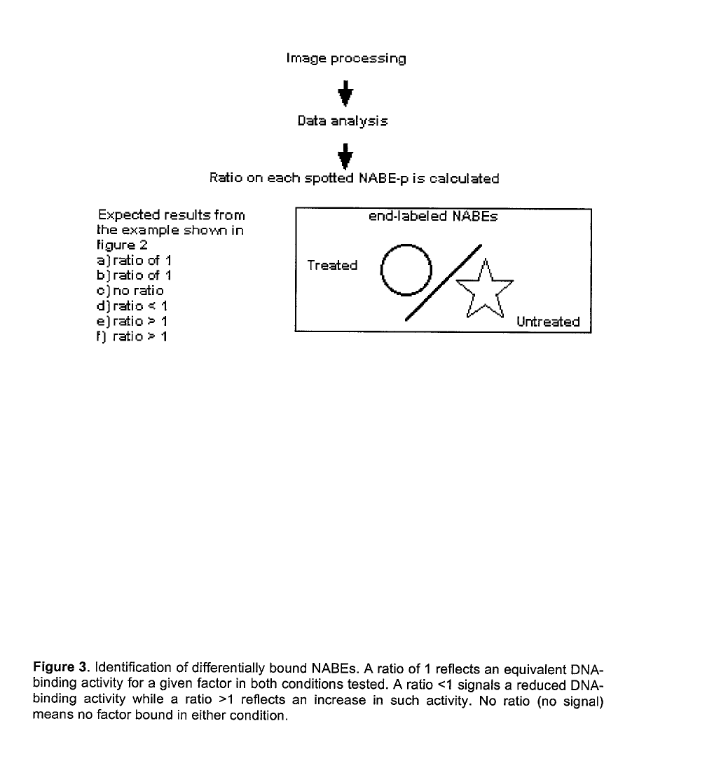

Differential NABE or NABF activity is assessed by scanning the hybridized microarray using an appropriate microarray scanning system. In a typical experiment, Cy3 and Cy5 fluorescent dyes are used to label NABEs. In this case, a scanner capable of detecting both the Cy3- and Cy5- labelled probes is used to produce TIFF images for each dye. With the appropriate software, images are analyzed to integrate the fluorescent intensity into pixels. These numerical values allow the determination of the relative quantity of each NABE (see FIG. 3; ratio measurement).

The data generated by the hybridization to the microarrayed NABE-ps must be further refined before differential NABE or NABF activity can be demonstrated. This process involves the normalization of the relative fluorescent intensities in each of the two scanned channels (Cy3 is scanned in a green channel, while Cy5 is read in a red channel). Normalization is necessary to adjust for differences in labelling and detection efficiencies for the fluorescent labels, as well for differences in the quantity of starting material from the two samples examined in the assay. These problems can cause a shift in the average ratio of Cy5 over Cy3 and the intensities must be readjusted before an experiment can be properly analyzed. Depending on the experimental design, several approaches may be used to calculate the normalization factor. A simple way makes use of the total measured fluorescent intensity. The assumption underlying this approach is that the total NABEs labelled with either Cy3 or Cy5 is quantitatively equal. While the intensity for any one spot may be higher in one channel than the other, when averaged over hundreds of spots in the array, these fluctuations should average out. Consequently, the total integrated intensity across all the spots in the array should be equal for both channels. A second approach can use as standards the non-regulated NABE-NABF complexes that should be detected in equal amounts in both samples. An EMSA assay (with ³²P-labelled probe) can be performed in order to demonstrate that there is no variation in NABE-NABF complex formation for some NABEs. In this case, the ratio of measured Cy5 to Cy3 ratios for these NABEs can be taken into account and the mean of the ratio adjusted to 1.

Results obtained from the following examples demonstrate that NABE-p arrays can be used for the specific detection of purified NABEs and that it can be used to demonstrate differential DNA-binding activity in two different samples.

### EXAMPLE 1

### Perfect Match Versus Mismatch NABE-p To Assess Hybridization Specificity Of NABE

Step 1: Microarray fabrication

In the inventors' experiments, arraying was performed using a SDDC-2 apparatus (ESI, Inc.). Printing was performed using an ArraylT™ Stealth Micro Spotting Technology (developed by TeleChem International, Inc.; FIG. 1). All procedures were performed in HEPA filter-regulated and humidity-controlled clean room environments. 3D-Link Activated slides from Surmodics Inc. were used according to the supplier's protocol for covalent attachment of the 5-amino modified oligonucleotides.

Table 1 gives the list of the arrayed NABE-ps. All these spotted oligonucleotides have 5-amino C-12 modifications and were synthesized by Cortec DNA Service Laboratories Inc., Kingston, Canada. For each NABE-p, a mutant or mismatch NABE-p MUT was also synthesized and spotted.

Step 2: NABE labelling

NABEs used for this experiment are listed in Table 2. The inventors used commercially available NABEs (Geneka Biotechnology Inc.). Two (2) ng of each of these double-stranded oligonucleotide were pooled and desalted before labelling. The desalting step was performed according to the supplier's protocol for the MicroSpin G-25 desalting column (Pharmacia Amersham). NABEs were resuspended in 12 μl of water. Labelling was carried out under the following conditions. The NABEs were heated at 95° C. for 3 minutes to obtain single-stranded DNA and then put on ice. The NABEs were mixed with 5 μl of 5×reaction buffer (1 mM potassium cacodylate, 125 mM Tris-HCl pH 6.6, BSA 1.25 mg/ml), 4 μl of 5 mM CoCl2, 3 μl of 1 mM of Cy5 dCTP (Amersham Pharmacia) and 1 μl of terminal transferase (25 U/μl, Amersham Pharmacia) for a final volume of 25 μL. The reaction was incubated at 37° C. for 1.5 hour. The unincorporate nucleotides were removed using the GFX PCR DNA and Gel Band Purification Kit (Amersham Pharmacia) following the supplier's protocol. The end-labelled NABEs were eluted in 50 μl of water. The samples were evaporated and resuspended in 20 μl of water. The samples were kept at −20° C. until use.

Step 3: Hybridization

In the inventors' examples, the blocking step for the slides was performed according to the slide supplier's instructions (Surmodics Inc.). For the hybridization mixture, preheated (95° C., 3 min) labelled NABEs were combined with 1 volume of the hybridization solution (50% formamide, 10×SSC, 0.2% SDS), and with 50 μg of herring sperm DNA. The hybridization mixture was added on the slide (40 μl final), covered with a polyethylene hydrophobic cover slip (PGC Scientific) and placed in a sealed hybridization chamber (Corning Costar) for an overnight incubation at 42° C. The array was then removed from the hybridization chamber, placed in a staining dish containing low-stringency wash buffer and washed according to standard procedure.

Step 4: Data analysis

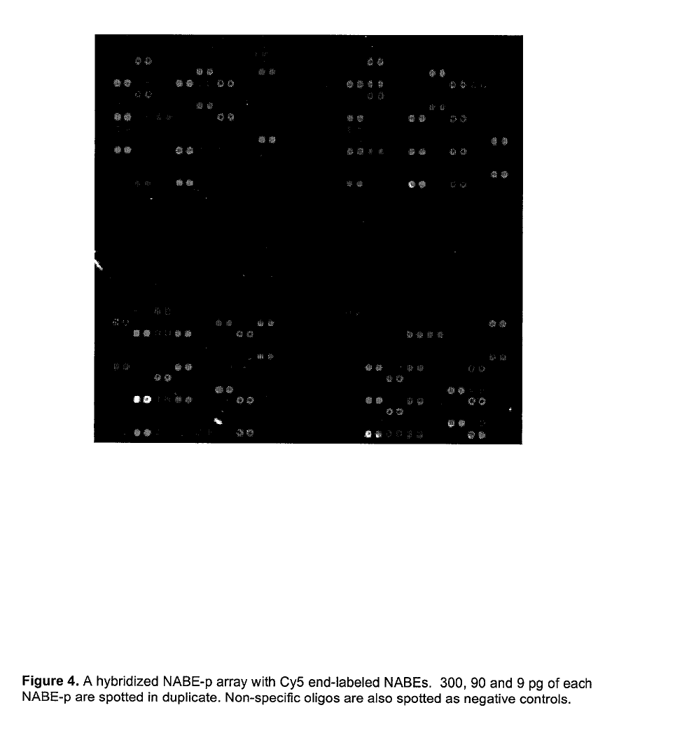

The inventors used the GenePix4000 (Axon Inc.) laser scanner to read the Cy5 hybridized NABE on the microarray (see FIG. 4). The intensity of each spot image was calculated using the GenePix version 2.0 software (Axon Inc.).

The results shown in FIG. 4 and summarized in Table 3 demonstrate the hybridization specificity of NABEs on a NABE-p array. First, a decrease in signal intensity for each NABE-p MUT was observed compared with the corresponding NABE-p with but one exception, for the YY1 NABE (MUT/WT %=100%). This widespread decrease in intensity reflects a lower level of hybridization of the NABEs to the mutant probes than to the wild type (wt) probes. For a specific hybridization, this is the expected result since mismatches between NABE-p MUT and NABEs cause destabilization and a lower melting temperature in the resulting hybrid. In some cases, the mismatch goes so far as to cause the abolition of the signal. In the case of YY1, the explanation for the lack of decrease in signal could be the length of the oligo, the YY1 oligo being exceptionally longer than the other oligos used in the assay. Table 1 points out the longer size of the YY1 oligo.

In this experiment, the inventors could also observe a lack of signal with the Oct NABE. This is explained by the fact that the inventors voluntarily omitted the Oct NABE in the labelling reaction in order to have a negative control for the hybridization. The absence of signal on both the Oct NABE-p and Oct NAB-p Mut is another indication of the hybridization specificity.

### EXAMPLE 2

### Hybridization Specificity Of Each NABE On NABE-p Array

In this example, four hybridization experiments were performed as in Example 1, with the exception that Cy3-dCTP was used as a labelling agent and some NABEs were volontarily omitted from each NABE-labelling mixture. For these experiments, and unlike what occurred in Example 1, Oct NABE was present is each labelling reaction. Results for this Oct NABE-p were used to normalize results from each experiment in order to obtain a similar Oct intensity.

Experimental results are shown in Table 4. They clearly demonstrate that the detected signal on any NABE-p comes from the corresponding labelled NABE. When one NABE is absent from the labelling mixture, no signal is detected on its cognate NABE-p (the signal being below the set threshold for a significant result). The one exception to this rule was with the Sp1 NABE (Exp. 3), which showed signal intensities above the threshold of 1000 pixels. This would mean that some end-labelled NABEs are non-specifically bound to the Sp1 NABE-p. However, the very high hybridization intensities of Sp1 NABE from the other experiments render the contribution of such non-specific hybridization negligible.

### EXAMPLE 3

### NABEs Isolated On Account Of Their Actual Binding To Transcription Factors Can Be Specifically Identified By The NABE-p Microarray

A pool of NABEs containing binding sites for the transcription factors c-Rel, E2F-1, Egr-1, ER, NFκB p50, p53, Sp1 and YY1 was incubated in a pool of nuclear protein extract coming from Raji, MCF-7 and K562 cell lines. All the material was provided by Geneka Biotechnology Inc. and the different components were incubated together as if to perform an EMSA experiment, as per the supplier's protocols. The DNA-protein complexes were then submitted to electrophoresis on a non-denaturing, 0.5×TGE, 5% polyacrylamide gel in order to separate the slower-migrating complexes from the free DNA elements. Markers consisting of a DNA-protein complex involving a ³²P-labelled Oct 1 probe (supplied by the same provider) and the same nuclear extract mix were deposited on the same gel, flanking the wells containing the experimental mixture. After the gel had been run long enough to allow the separation of complexed from free DNA, the migration was stopped, one of the plates containing the gel removed, and the remaining plate supporting the gel was wrapped in ordinary kitchen plastic wrap. The gel was then deposited in the dark on an X-ray film, its position marked with a pen. It was left to expose the film for a short period, just enough to allow the labelled DNA present in the gel to be localized. The film was developed and used to properly align the gel so that a band of acrylamide containing the unlabelled DNA-protein complexes could be sliced out (the band being located in between the two shifted bands revealed by the X-ray film exposure).

The acrylamide slice was introduced with 500 μl TAE buffer (20 mM Tris, 10 mm CH₃COOH, 50 mm EDTA, pH 8) in a length of Spectra/Por 7000 dialysis tubing (Spectrum Laboratories, Inc.), the end of which were then clipped shut, and submitted to a brief electroelution (50 volts, 15 minutes) to allow the DNA to migrate out of the acrylamide matrix and into the buffer. The buffer was then recovered from the tube, purified by phenol-chloroform extraction as per the usual protocols, adjusted to a final CH₃COONa concentration of 0.3M and precipitated by addition of one volume isopropanol.

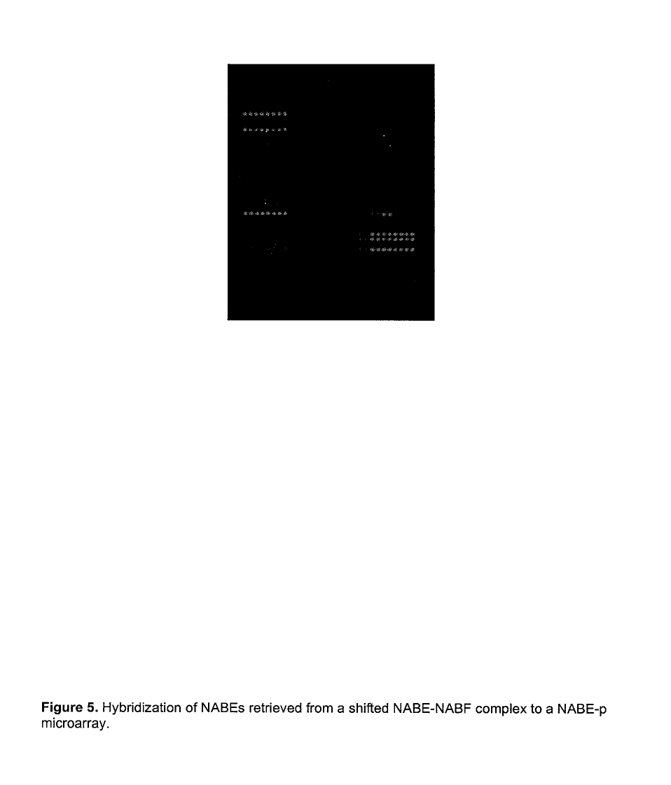

The DNA pellet was then resuspended in water and labelled with Cy5 as described above. It was then analyzed by NABE-p microarray. The results, which may be seen in FIG. 5, reveal that all the NABEs used in the experiment are specifically detected by the NABE-p microarray. One of the NABEs, p53, gives a much weaker signal than the others; this is actually an expected result, since the supplier does show that the combination of this particular NABE and the cell line (Raji) used to provide the NABF (p53) results in a weak interaction (Geneka Biotechnology Inc. 2000 catalog, p. 194, FIG. 1). No other NABE-p was bound by the NABEs obtained from the electroeluted material, demonstrating the specificity of the signals.

### EXAMPLE 4

### The DNA-proteomic Array Detects Differential DNA-binding Activity In Two Independent Nuclear Extracts

New DNA-protein interaction reactions were set up as above, using NABEs for the transcription factors AP-1, c-Rel, Egr-1, NFκB p50, Sp1, USF and YY1. The nuclear extracts to be compared in terms of DNA-binding activity were made from Jurkat cells and Jurkat cells treated with TPA on the one hand, and K562 cells and K562 cells treated with TPA on the other hand. Since electrelution and later manipulation can introduce a bias in the comparison between the two samples, a recovery control was added to these experiments: a NABE with the binding site of the estrogen receptor ER was independently incubated with an extract from the breast carcinoma cell line MCF-7. When the complexes formed by the incubation of NABEs in nuclear extracts from treated and untreated cells were loaded on two different gels, an equal amount of the ER/MCF-7 extract reaction was added next to each in a neighbouring well. Since the amount of shifted material from ER/MCF-7 reaction must be the same in each gel, the microarray analysis results of the two reactions (with TPA and without TPA) can be normalized using the ER NABE-p result as a standard.

The DNA from each shifted band was electroeluted and purified as described above. The DNA derived from complexes formed in nuclear extracts made from untreated cells was labelled with Cy3, while the DNA obtained from complexes formed in nuclear extracts made from treated cells was labelled with Cy5. The labelled DNAs were then co-hybridized to the NABE-p microarray. If the treatment causes a decrease in DNA-binding activity for a particular transcription factor, the Cy3-labelled NABEs should dominate and cause a green color to appear at the appropriate point. If, on the other hand, the treatment has caused an increase in such activity, the Cy5-labelled NABEs should dominate and give the same spot a red color. Equal hybridization of NABEs coming from the two experiments should average out and give the spot a yellow color. The GenePix version 2.0 software (Axon Inc.) can quantify the green and red pixels and provide quantifiable values for each hybridization.

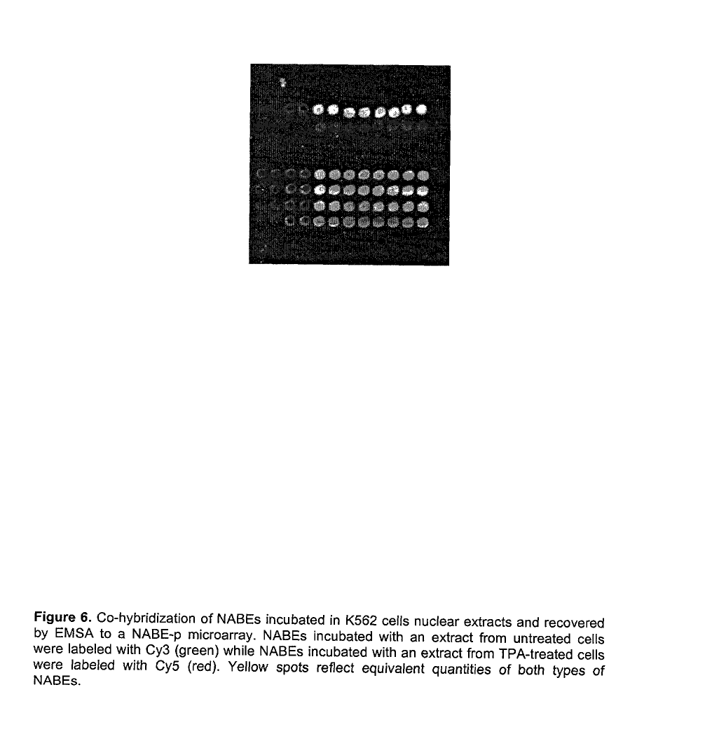

FIG. 6 shows the co-hybridization of NABEs purified from DNA-protein complexes obtained from nuclear extracts of K562 cells treated with the phorbol ester TPA or left untreated. The treated NABEs were labelled with Cy5, while the untreated ones were labelled with Cy3. It can clearly be seen that while some factors remained unaltered in their DNA-binding activity, others were clearly influenced by the TPA treatment and saw their activity either increase or decrease.

Although the present invention has been described hereinabove by way of preferred embodiments thereof, it can be modified, without departing from the spirit and nature of the subject invention as defined in the appended claims.

TABLE 1
Nucleotide sequence of NABE-probes used to make
DNA microarrays.

| | |
|---|---|
| sequence | NABE-p name |

| | |
|---|---|
| CGCTTGATGAGTCAGCCGGAA | AP-1 |
| CGCTTGATGACCCAGCCGGAA | AP-1 MUT |
| CCACAAACGACCGCCCGCGGGCGGT | AP-2 |
| CCACAAACGACCGATTGCGGGCGGT | AP-2 MUT |
| GATTCAATGACATCACGGCTGTG | ATF-2 |
| GATTCAAGAACATAGCGGCTGTG | ATF-2 MUT |
| AGCTTGGGGTATTTCCAGCCG | c-Rel |
| AGCTTGGCATAGGTCCAGCCG | c-Rel MUT |
| GGTTTGTGTTTAGGCGCGAAAACTGAA | E2F-1 |
| GGTTTGTGTTTAGGTACGAAAACTGAA | E2F-1 MUT |
| GGATCCAGCGGGGGCGAGCGGGGGCGAACG | Egr-1 |
| GGATCCAGCGGGTACGAGCGGGTACGAACG | Egr-1 MUT |
| TAATAGGTCACAGTGACCTGATTCC | ER |
| TAATACCGCACAGTGAAATGATTCC | ER MUT |
| GCCATGGGGGGATCCCCGAAGTCC | NFkB p50 |
| GCCATGGGCCGATCCCCGAAGTCC | NFkB p50 MUT |
| CCTCTTGGATTTGCATATGGGCTG | Oct |
| CCTCTTGGATGATTATATGGGCTC | Oct MUT |
| AGCTGGACATGCCCGGGCATGTCC | p53 |
| AGCTGGATCGCCCCGGGCATGTCC | p53 MUT |
| GTCGACATTTCCCGTAAATCGTCGA | SIE |
| GTCGACATATAGCGTAAATCGTCGA | SIE MUT |
| CCCTTGGTGGGGGCGGGGCCTAAGCTGCG | Sp1 |
| CCCTTGGTGGGTTGGGGGCCTAAGCTGCG | Sp1 MUT |
| GGCCAGACCACGTGGTCTGTTC | USF |
| GGCCAGACACAGTGGTCTGTTC | USF MUT |
| GGGGATCAGGGTCTCCATTTTGAAGCGGGATCTCCC | YY1 |
| GGGGATCAGGGTCTTTGTTTTGAAGCGGGATCTCCC | YY1 MUT |

For each NABE-p containing a consensus binding site for a transcription factor, a corresponding NABE-p MUT was synthesized, containing a mutated consensus site, each MUT sequence differing from the normal sequence by 1 to 4 nucleotides. The sequence of each of these NABE-p is complementary to one strand of the corresponding NABE used in the binding assay.

TABLE 2

Nucleotide sequence of the double stranded NABE
that contain DNA

| | |
|---|---|
| Double stranded oligo sequences | name |

| | |
|---|---|
| 5′-CGCTTGATGAGTCAGCCGGAA-3′ | AP-1 |
| 3′-GCGAACTACTCAGTCGGCCTT-5′ | |
| 5′-CCACAAACGACCGCCCGCGGGCGGT-3′ | AP-2 |
| 3′-GGTGTTTGCTGGCGGGCGCCCGCCA-5′ | |
| 5′-GATTCAATGACATCACGGCTGTG-3′ | ATF-2 |
| 3′-CTAAGTTACTGTAGTGCCGACAC-5′ | |
| 5′-AGCTTGGGGTATTTCCAGCCG-3′ | C-Rel |
| 3′-TCGAACCCCATAAAGGTCGGC-5′ | |
| 5′-GGTTTGTGTTTAGGCGCGAAAACTGAA-3′ | E2F-1 |
| 3′-CCAAACACAAATCCGCGCTT TTGACTT-5′ | |
| 5′-GGATCCAGCGGGGGCGAGCGGGGGCGAACG-3′3′- | Egr-1 |
| CCTAGGTCGCCCCC GCTCGCC CCCGCTTGC-5′ | |
| 5′-TAATAGGTCACAGTGACCTGATTCC-3′ | ER |
| 3′-ATTATCCAGTGTCACTGGACTAAGG-5′ | |
| 5′-GCCATGGGGGGATCCCCGAAGTCC-3′ | NFkB p50 |
| 3′-CGGTACCCCCCTAGGGGCTTCAGG-5′ | |
| 5′-CCTC TTGGATTTGCATATGGGCTG-3′ | Oct |
| 3′-GGAGAACCTAAACGTATACCCGAC-5′ | |
| 5′-AGCTGGACATGCCCGGGCATGTCC-3′ | p53 |
| 3′-TCGACCTGTACGGGCCCGTACAGG-5′ | |
| 5′-GTCGACATTTCC CGTAAATCGTCGA-3′ | SIE |
| 3′-CAGCTGTAAAGGGCATTTAGCAGCT-5′ | |
| 5′-CCCTTGGTGGGGGCGGGGCCTAAGCTGCG-3′ | Sp1 |
| 3′-GGGAACCACCCCCGCCCCGGATTCGACGC-5′ | |
| 5′-GGGGATCAGGGTCTCCATTTTGAAGCGGGATCTCCC- 3′ | YY1 |
| 3′-CCCCTAGTCCCAGAGGTAAAACTTCGCCCTAGAGGG-5′ | |

binding consensus sequence. The name of each NABE correspond to NABF or family of NABF that bound to the consensus sequence.

TABLE 3

| | 300 pg of spotted NABE-p | | | 90 pg of spotted NABE-p | | |
|---|---|---|---|---|---|---|
| NABE name | WT | MUT | MUT/WT (%) | WT | MUT | MUT/WT (%) |
| AP-1 | 21197 | 7584 | 36 | 20964 | 5070 | 24 |
| AP-2 | 21772 | 9261 | 43 | 19227 | 5978 | 31 |
| ATF-2 | 6988 | 187 | 3 | 3520 | 371 | 11 |
| c-Rel | 7715 | 148 | 2 | 6024 | 257 | 4 |
| E2F-1 | 10458 | 4174 | 40 | 7062 | 4147 | 59 |
| EGR-1 | 23940 | 13931 | 58 | 21861 | 9332 | 43 |
| ER | 32317 | — | — | 17909 | 189 | 1 |
| NFkBp50 | 65535 | 15301 | 23 | 41665 | 19127 | 46 |
| Oct | 151 | 133 | 88 | 162 | 168 | 104 |
| p53 | 40943 | 9180 | 22 | 35144 | 10548 | 30 |
| Sp1 | 65535 | 35436 | 54 | 54226 | 31644 | 58 |
| YY1 | 20136 | 16958 | 84 | 19236 | 22848 | 119 |
| Results from a NABE-p array hybridized with certain Cy5-end-labeled NABEs shown in FIG. 4. Values correspond to median pixel intensities detected by the scanner at a wavelength of 635 obtained for the corresponding NABE-p. WT refers to Wild-type or normal NABE-p sequences, MUT refers to the mutant NABE-p. MUT/WT (%) represent the percentage of the intensities on the MUT NABE-p relative to the corresponding WT NABE-p. In this experiment the median background pixel intensities are less than 113 pixels. | | | | | | |
| # Oct NABE was voluntarily omitted from the labeling reaction in order to have a negative hybridization control. | | | | | | |

TABLE 4

| | EXP 1 | EXP 2 | EXP 3 | |
|---|---|---|---|---|
| | AP-1 | AP-2 | ATF | EXP 4 |
| | E2F | ER | NFkB | c-Rel |
| Missing NABE | Egr-1 | YY1 | Sp1 | p53 |
| NABE Name | Intensities | Intensities | Intensities | Intensities |
| AP-1 | 221 | 7 566 | 4 844 | 1 836 |
| AP-2 | 18 113 | 844 | 10 344 | 4 488 |
| ATF-2 | 5 057 | 7 382 | 134 | 2 518 |
| c-Rel | 10 799 | 14 537 | 10 657 | 80 |
| E2F-1 | 28 | 10 502 | 14 544 | 5 842 |
| Egr-1 | 44 | 13 534 | 14 245 | 7 454 |
| ER | 10 476 | 33 | 9 051 | 7 576 |
| NFkBp50 | 10 590 | 16 984 | 316 | 5 735 |
| Oct | 4 127 | 4 089 | 4 100 | 4 110 |
| p53 | 17 018 | 21 191 | 73 217 | 204 |
| Sp1 | 41 198 | 47 672 | 1 761 | 21 326 |
| YY1 | 12 423 | 245 | 8 160 | 9 155 |

Results from 4 hybridization experiments (EXP 1 to 4) of cy3-end labeled-NABE on NABE-probe array. In each experiment some NABE was voluntary omit from the labeling mixte (identify by the gray box) in order to demonstrate the specificity of hybridization of each NABE on the corresponding NABE-p. In this experiment the median background pixel intensities is lower then 255 pixels.

List of References

1. Ausubel, F. M. et al., “Current Protocol in Molecular Biology”, New York, Greene and Wiley, 1993, pp.12.2.1-12.2.5.

2. Egholm M, Buchardt O, Christensen L, Behrens C, Freier S M, Driver D A, Berg R H, Kim S K, Norden B, Nielsen P E.(1993) Nature 365: 566-8

3. Furlow, J. D., et al., (1993) High affinity binding of the estrogen receptor to a DNA response element does not require homodimer formation or estrogen. J. Biol. Chem. 268:12519.

4. Glass, C. K., et al., (1987) A c-erb-A binding site in rat growth hormone gene mediates trans-activation by thyroid hormone. Nature 329 :738.

5. Ludwig, L. B., et al., (1990) A microtiter well assay for quantitative measurement of estrogen receptor binding to estrogen-responsive elements. Mol. Endocrinol. 4 :1027.

6. Orum H, Nielsen P E, Egholm M, Berg R H, Buchardt O, Stanley C.(1993) Nucleic Acids Res. 21: 5332-6

7. Peale, F. V. et al., (1988) Properties of a high-affinity DNA binding site for estrogen receptor. Proc Natl Acad Sci USA 85 :1038.

8. Ren, B., et al., (2000) Genome-Wide Location and Function of DNA Binding Proteins. Science 290 :2306.

9. Shupnik, M. A. and Rosenzweig, B. A. (1991) Identification of an estrogen-responsive element in the rat LH beta gene. DNA-estrogen receptor interactions and functional analysis. J. Biol. Chem. 266 :17084.

10. Suske, G., et al., (1989) Non-radioactive method to visualize specific DNA-protein interactions in the band shift assay. Nucleic Acids Res. 17 :4405.

11. Uhlman, E. and Peyman, A. (1990). Antisense oligonucleotides: A new therapeutic principle. Chemical Rev. 90, pp. 545-569.

12. U.S. Pat. No. 6,100,035.

What is claimed is:

1. A method for screening nucleic acid binding elements (NABEs), said method comprising:
(a) contacting nucleic acid binding factors (NABFs) with NABEs under conditions to promote specific binding interactions therebetween;
(b) identifying complexes formed between said NABEs and said NABFs (NABE-NABF complexes);
(c) separating said NABF from said NABE-NABF complexes to obtain NABEs that bind to NABFs;
(d) marking said NABEs obtained in (c) (marked NABEs);
(e) contacting said marked NABEs with probes of known nucleic acid binding elements (NABE-ps) bound to a support under conditions to promote hybridization therebetween; and
(f) analyzing the hybridization in (e) in order to identify said marked NABEs.

2. A method according to claim 1, wherein said NABEs are extracted from a cell.

3. A method for screening nucleic acid binding elements (NABEs) that are differentially active in modified cells, said method comprising:
(a) contacting nucleic acid binding factors (NABFs) with NABEs derived from a modified cell under conditions to promote specific binding interactions therebetween;
(b) identifying complexes formed in (a) between said NABEs and said NABFs (NABE-NABF complexes);
(c) separating said NABFs from said NABE-NABF complex to obtain NABEs that bind to NABFs;
(d) marking said NABEs obtained in (c) (marked NABEs);
(e) contacting said marked NABEs with probes of known nucleic acid binding elements (NABE-ps) bound to a support under conditions to promote hybridization therebetween; and
(f) analyzing the hybridization in (e) in order to identify said marked NABEs that are differentially active in the modified cell.

4. A method according to claim 3, further comprising
(g) contacting said NABFs with nucleic acid binding elements derived from a non-modified cell (non-modified cell NABEs) under conditions to promote specific binding interactions therebetween;
(h) identifying complexes formed in (i) between said non-modified cell NABEs and said NABFs (non-modified cell NABE-NABF complexes);
(i) separating said NABF from said non-modified cell NABE-NABF complex to obtain non-modified cell NABEs that bind with NABFs;
(j) marking said non-modified cell NABEs obtained in (i) with a marker that is different than the marker used to mark said modified cell NABEs (marked non-modified cell NABEs);
(k) contacting said marked non-modified cell NABEs with said NABE-ps bound to a support under conditions to promote hybridization; and
(l) comparing the amount of hybridization in (v) with the amount of hybridzation in (e) in order to identify the differentiated activity of said marked NABEs derived from the modified cell.

5. A method according to any one of claims 1-4, wherein said contacting in (a) includes incubating a pool of said NABEs with a pool of said NABFs in conditions conducive to the formation of said NABE-NABF complexes.

6. A method according to any one of claims 1-4, wherein said identification in (b) includes submitting said contacted NABFs and NABEs in (a) to an electrophoresis separation.

7. A method according to claim 6, wherein said electrophoresis separation is part of an electromobility shift assay (EMSA).

8. A method according to any one of claims 1-4, wherein said separation in (c) includes purification of said NABE-NABF complexes in order to eliminate the NABFs bound to said NABEs.

9. A method according to any one of claims 1-4, wherein said marking in (d) includes labelling said NABEs with a terminal primer marker.

10. A method according to claim 9, wherein said terminal primer marker is a fluorescent or radioactive label.

11. A method according to any one of claims 1-4, wherein said NABE-ps are bound to a microarray or microbeads.

12. A method according to any one of claims 1-4, wherein said NABEs contain binding sites for transcription factors.

13. A method as defined in claim 12, wherein said transcription factors include c-Rel, E2F-1, Egr-1, ER, NFκB p50, p53, Sp1 and YY1.
